(19) United States
(12) Patent Application Publication
(10) Pub. No.: US 2023/0089605 A1
(43) Pub. Date: Mar. 23, 2023

(54) USE OF DOXYCYCLINE, RESVERATROL, IVERMECTIN, AND RELATED CHEMICALLY MODIFIED COMPOUNDS TO TREAT OR PREVENT VIRAL INFECTIONS

(71) Applicant: Ardestat Biologics, LLC, Atlanta, GA (US)

(72) Inventor: Howard Tenenbaum, Toronto (CA)

(21) Appl. No.: 17/932,343

(22) Filed: Sep. 15, 2022

Related U.S. Application Data
(60) Provisional application No. 63/246,049, filed on Sep. 20, 2021.

(51) Int. Cl. A61K 31/65; A61K 31/05; A61K 31/7048; A61K 31/616; A61K 35/16; A61P 31/14; A61K 45/06; A61K 31/56

(52) U.S. Cl. CPC A61K 31/65; A61K 31/05; A61K 31/7048; A61K 31/616; A61K 35/16; A61P 31/14; A61K 45/06; A61K 31/56

(57) ABSTRACT

In accordance with the purpose(s) of the present disclosure, as embodied and broadly described herein, the disclosure, in one aspect, relates to a therapeutic treatment or prevention of viral infections such as, for example, COVID-19. In one aspect, the therapeutic treatment combines the administration of doxycycline and/or chemically-modified tetracyclines with resveratrol and ivermectin. In another aspect, the disclosed treatment offers anti-inflammatory effects, can combat the effects of reactive oxygen species, and can inhibit the activity of matrix metalloproteinase enzymes; these combined effects may, in some aspects, be useful in preventing or mediating the effects of the cytokine storm that is associated with poor COVID-19 outcomes including death. In some aspects, dosages of the treatment can be modified to account for COVID-19 comorbidities including, but not limited to, such conditions as diabetes or hypertension.

### CROSS-REFERENCE TO RELATED APPLICATIONS

This application claims the benefit of and priority to co-pending U.S. Provisional Patent Application No. 63/246,049, filed on Sep. 20, 2021, the contents of which are incorporated by reference herein in their entireties.

### BACKGROUND

The outbreak of viral pneumonia cases related to SARS-CoV-2 was first reported by Chinese health authorities in December 2019. SARS-CoV-2 is an enveloped, positive-sense single-stranded RNA virus that binds to the angiotensin converting enzyme 2 (ACE2) receptor in order to gain entry into host cells through its transmembrane spike (S) glycoprotein. As with other viral infectious entities, SARS-CoV-2 can lead to various respiratory infections, including multifocal interstitial pneumonia, which represents the most common cause of admission to intensive care and death in patients infected with SARS-CoV-2 (leading to the disease now referred to as COVID-19). This infection triggers the development of the often fatal acute respiratory distress syndrome (ARDS), a condition that is characterized not only by respiratory failure but also by the development of thromboembolic disease. Similarly, secondary infection by influenza viruses is also associated with a greater risk to develop bacterial pneumonia. This association was described initially in reference to the observed increase of pneumonia cases during the epidemic of influenza in Sheffield UK in 1803 and after the influenza pandemic in 1918. Haemophilus influenzae has been identified as one of the etiologic bacterial agents that cause pneumonia and has been implicated in the etiology of community-acquired pneumonia (CAP) along with other bacterial infections.

ARDS has been associated with influenza and coronavirus infections as well as bacterial pneumonia, and although its precise pathophysiologic mechanism remains largely unknown, it is actually a result of direct damage caused by the viral or bacterial pathogen followed by the triggering of severe immunologic injury caused by an extreme host immune response, and ultimately, by the production of massive amounts of dead cellular debris in the circulation that triggers further a hyperinflammatory response (this explains the delay often seen in the development of ARDS in relation to when initial infection has occurred). More importantly, it has been argued that host-derived damage control rather than viral burden alone is the cause of respiratory viral and/or bacterial infection-induced morbidity and mortality associated with a massive infiltration of polymorphonuclear neutrophils (PMNs) in the lungs. Despite playing a central role in the resolution of inflammation, they can also become “hyperactive,” and when this happens these cells produce high levels of pro-inflammatory cytokines, high levels of reactive oxygen species (ROS), and very high and damaging levels of matrix metalloproteinases (MMP) and elastase that lead to a dysregulated and deleterious pro-inflammatory state within the lungs and other organs in patients, i.e. ARDS. The degradative enzyme groups referred to herein play an important role in the damage of lung tissues as well as deleterious changes observed in patients with ARDS including damage to other organ systems (i.e. not only lung) including the vasculature. Paradoxically then, despite their critical played by PMN cells insofar as clearance of viral pathogens as well as management of infectious disease in general are concerned overly sensitized/activated PMN responses can lead to a vicious inflammatory cycle leading eventually to damage of the very tissues that they were dispatched to protect in the first place but as a consequence of the so-called “cytokine storm” mediated by PMNs.

Currently, the emergence of SARS-CoV-2 infection has resulted in a high number of hospitalizations, with many of those hospitalized ultimately developing ARDS at different levels of severity. However, while an increase in circulating mature and immature neutrophil populations is observed in both COVID-19 and non-COVID-19 ARDS, an exaggerated neutrophil activation and degranulation, as well as formation of neutrophil platelet aggregates and activation of the clotting cascade are observed in COVID-19 ARDS only as mentioned above in relation to thromboembolic disease. In terms of treatment, whilst dexamethasone has been observed to decrease the immature neutrophil population, no effect has been observed when a prothrombotic hyperinflammatory neutrophil state is in place.

Every time a new respiratory viral disease emerges such as severe acute respiratory syndrome coronavirus-2 (SARS CoV-2) there is a potential for worldwide spread that can cause millions of deaths, in this case amongst SARS CoV-2 naïve individuals and in the case of this or any novel virus this effectively represents a majority of the population. Amongst all medical and public health countermeasures, vaccination is the most effective strategy to mitigate the devastating impact of viral diseases on the population. However, the administration of a vaccine in the midst of a pandemic can actually drive viral mutation, and is generally reserved for use prior to the advent of any particular seasonal virus. Moreover, even if there was a perceived need to vaccinate people during a pandemic, the time until the production of a well-matched, safe, and effective vaccine for a new disease can lead to more lives lost and their effectiveness is usually compromised by continuous viral mutations and evolution of major antigens in the long term rendering some vaccines ineffective over time.

Therefore, there is a critical need to be able to treat respiratory virus related diseases and this can only come by way of the development of novel and effective therapeutics, and also while following the principles that 1) treatment must be initiated as soon as possible after the development of symptoms, and 2) that no single drug can be relied upon to manage COVI D-19 disease given the staged and complex nature of this condition. Since SARS-CoV-2 targets cells in any tissue or organ expressing the receptor for ACE2, COVI D-19 has been seen as a systemic infection. Moreover, since the pathogenesis of COVID-19 takes advantage of an early and unbalanced immune response, the most effective intervention should modulate the host immune response during the incipient stages of infection, as the most critically ill and deceased patients do not usually manifest severe clinical symptoms in the early phases of the disease. In addition, and if also possible, such intervention should interfere with viral replication so that the actual spread of viral particles in an infected individual can be blocked. These needs and other needs are satisfied by the present disclosure.

### SUMMARY

In accordance with the purpose(s) of the present disclosure, as embodied and broadly described herein, the disclosure, in one aspect, relates to a therapeutic treatment or prevention of viral infections such as, for example, COVID-19. In one aspect, the therapeutic treatment combines the administration of doxycycline and/or chemically-modified tetracyclines with resveratrol and ivermectin. In another aspect, the disclosed treatment offers anti-inflammatory effects, can combat the effects of reactive oxygen species, and can inhibit the activity of matrix metalloproteinase enzymes; these combined effects may, in some aspects, be useful in preventing or mediating the effects of the cytokine storm that is associated with poor COVID-19 outcomes including death. In some aspects, dosages of the treatment can be modified to account for COVID-19 comorbidities including, but not limited to, such conditions as diabetes or hypertension.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic representation of the interactions amongst and between four independent pathophysiologic pathways involved in the cytokine storm cascade during COVID-19. Mechanisms for COVID-19 parallel those described for periodontal diseases.

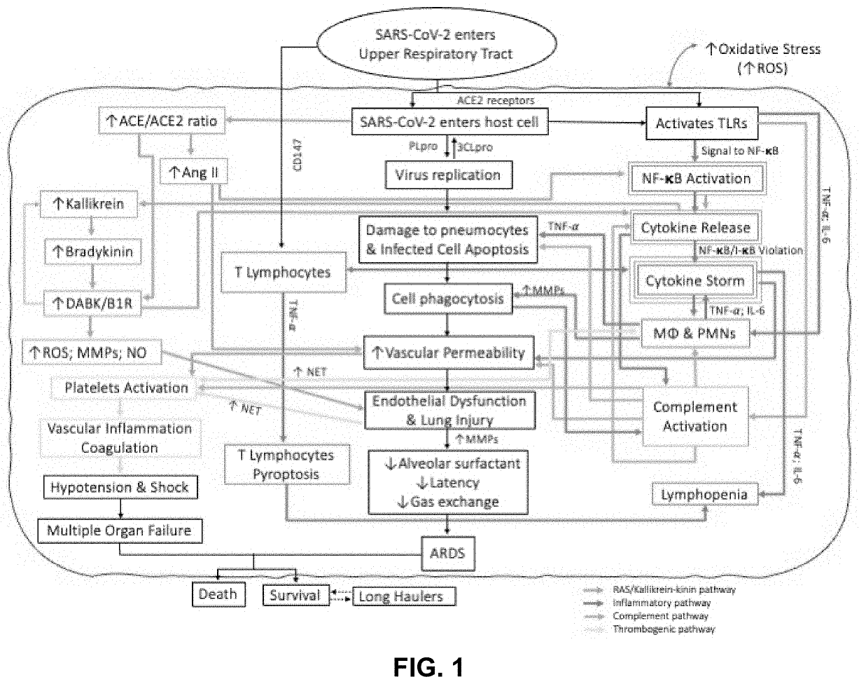

FIG. 2 shows that doxycycline synergizes with resveratrol to suppress MMP9 activity. Doxycycline, resveratrol, colchicine, and ivermectin were assessed at the indicated concentrations using the MMP9 Inhibitor Screening Assay Kit (abcam), according to the manufacturer's instructions.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a resveratrol isomer” “a chemically modified doxycycline,” or “an oral dosage form,” includes, but is not limited to, mixtures or combinations of two or more such resveratrol isomers, chemically modified doxycyclines, or oral dosage forms, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins;

mineral supplements; substances used for the treatment, prevention, diagnosis, cure or mitigation of disease or illness; or substances which affect the structure or function of the body; or prodrugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease or a symptom or condition thereof, such as acute respiratory distress syndrome (ARDS), inhibition of viral replication, and/or a cytokine storm. The effect can be therapeutic in terms of a partial or complete cure of a disease, condition, symptom or adverse effect attributed to the disease, disorder, or condition. The term “treatment” as used herein can include any treatment of ARDS and/or a cytokine storm in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder, or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1 -to-C 6 alkyl esters and C 5 -to-C 7 cycloalkyl esters, although C 1 -to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1 -to-C 6 alkyl amines and secondary C 1 -to-C 6 dialkyl amines. In the case of secondary amines, the amine can also be in the form of a 5- or 6-membered heterocycle containing one nitrogen atom. Amides derived from ammonia, C 1 -to-C 3 alkyl primary amides and C 1 -to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

Unless otherwise specified, temperatures referred to herein are based on atmospheric pressure (i.e. one atmosphere).

### Compounds

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid, and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quatemized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides, and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate, or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### Pharmaceutical Compositions

In one aspect, disclosed herein are pharmaceutical compositions including therapeutically effective amounts of a tetracycline and resveratrol. In another aspect, the pharmaceutical compositions further include ivermectin. In one aspect, the tetracycline can be doxycycline, a chemically-modified tetracycline, or any combination thereof. In one aspect, the chemically-modified tetracycline can be 6-demethyl-6-deoxy-4-dedimethylamino-tetracycline (CMT-3), 6a-deoxy-5-hydroxy-4-dedimethylamino-tetracycline (CMT-8), or a combination thereof. In another aspect, the resveratrol can be cis-resveratrol, trans-resveratrol, or a combination thereof.

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets, or tablets each containing a predetermined amount of the active ingredient, or can be suitable for administration by inhalation (e.g., nasal spray or nebulizer administration). Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymer; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate, and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate, and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe fur Pharmazie, Kostnetik and angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### Oral Dosage Forms

In one aspect, the pharmaceutical compositions comprise an oral dosage form. In another aspect, the oral dosage form comprises one or more tablets, capsules, or a combination thereof. In any of these aspects, the tetracycline and the resveratrol can be coformulated or can be separately formulated. In another aspect, the tetracycline and the resveratrol can be co-administered. In still another aspect, the ivermectin and the resveratrol can be coformulated or can be separately formulated. In another aspect, the ivermectin can be co-administered.

### Unit Doses for Active Ingredients

Doxycycline and Chemically-Modified Tetracyclines. In one aspect, the compositions, kits, and methods disclosed herein include doxycycline and/or one or more chemically-modified tetracyclines. In another aspect, the doxycycline and/or chemically-modified tetracyclines can be provided in capsules, powders to be reconstituted for IV administration, syrups, oral suspensions, tablets, delayed-release tablets, or any combination thereof. In another aspect, the dosage form can include an amount of doxycycline or chemically-modified tetracycline of from about 10 mg to about 300 mg, or of about 10, 20, 30, 40, 50, 60, 70, 75, 80, 90, 100, 110, 120, 130, 140, 150, 160, 170, 180, 190, 200, 210, 220, 230, 240, 250, 260, 270, 280, 290, or about 300 mg, or a combination of any of the foregoing values, or a range encompassing any of the foregoing values. In one aspect, the dosage form is a tablet including about 20, 30, 50, 60, 75, 100, 120, 150, or about 200 mg of doxycycline or chemically-modified tetracycline.

In one aspect, the doxycycline or chemically modified tetracycline can be administered for a period of from about 7 days to about 60 days, or for about 7, 10, 14, 21, 28, 35, 52, 49, 56, or about 60 days, or a combination of any of the foregoing values, or a range encompassing any of the foregoing values. In one aspect, the doxycycline or chemically modified tetracycline can be administered once per day, twice per day, or three times per day. In another aspect, the doxycycline or chemically modified tetracycline can be administered twice per day for an initial period followed by once per day for a maintenance period. In one aspect, the initial period can be one day, two days, or one week.

Resveratrol. In one aspect, the compositions, kits, and methods disclosed herein include resveratrol. In one aspect, the resveratrol is trans-resveratrol. In another aspect, the resveratrol is cis-resveratrol. In still another aspect, the resveratrol can be a mixture in any proportion of cis-resveratrol and trans-resveratrol. In another aspect, the dosage form including resveratrol can include an amount of resveratrol of from about 5 mg to about 3 g of resveratrol, or can be about 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, or about 950 mg, or can be about 1, 1.5, 2, 2.5, or about 3 g of resveratrol, or a combination of any of the foregoing values, or a range encompassing any of the foregoing values.

In one aspect, the resveratrol can be administered for a period of from about 7 days to about 60 days, or for about 7, 10, 14, 21, 28, 35, 52, 49, 56, or about 60 days, or a combination of any of the foregoing values, or a range encompassing any of the foregoing values. In one aspect, the resveratrol can be administered once per day, twice per day, or three times per day. In another aspect, the resveratrol can be administered twice per day for an initial period followed by once per day for a maintenance period. In one aspect, the initial period can be one day, two days, or one week. In some aspects, resveratrol can be administered for a longer period of time than the doxycycline or chemically-modified tetracycline. Further in these aspects, resveratrol can be administered for a period of from about 7 days to about 1 year, or for about 7 days, 30 days, 60 days, or about 3, 4, 5, 6, 7, 8, 9, 10, 11, or about 12 months, or a combination of any of the foregoing values, or a range encompassing any of the foregoing values.

Ivermectin. In one aspect, the compositions, kits, and methods disclosed herein include ivermectin. In a further aspect, the dosage form including ivermectin can include an amount of ivermectin of from about 5 mg to about 1 g, or can be about 5, 10, 15, 20, 25, 30, 35, 40, 45, 50, 100, 150, 200, 250, 300, 350, 400, 450, 50, 550, 600, 650, 700, 750, 800, 850, 900, or about 950 mg, or about 1 g, or a combination of any of the foregoing values, or a range encompassing any of the foregoing values. In one aspect, the amount of ivermectin is from about 18 mg to about 36 mg.

In one aspect, the ivermectin can be administered for a period of from about 7 days to about 60 days, or for about 7, 10, 14, 21, 28, 35, 52, 49, 56, or about 60 days, or a combination of any of the foregoing values, or a range encompassing any of the foregoing values. In one aspect, the ivermectin can be administered once per day, twice per day, or three times per day. In another aspect, the ivermectin can be administered 3-4 times per week for an initial period followed by once per week for a maintenance period. In one aspect, the initial period can be one day, two days, one week, or two weeks.

### Additional Therapeutic Agents

In an aspect, the kit can include one or more additional therapeutic agents such as, for example, acetylsalicylic acid. In a further aspect, the acetylsalicylic acid can be present in an amount of about 325 mg per unit dose. Without wishing to be bound by theory, inclusion of acetylsalicylic acid in the kits may assist in preventing adverse effects related to abnormal blood clotting and may additionally provide pain relief to ARDS patients.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for modulating cytokine activity (e.g., treatment of one or more symptoms associated with or effects of SARS-CoV-2 infection) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, and/or a polymorph thereof,. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional respiratory support therapeutic agent, anti-clotting therapeutic agent, an antibody treatment, or another useful therapeutic agent useful in COVID-19 treatment, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

### Kits

In a further aspect, the present disclosure relates to kits comprising the disclosed compounds, or a pharmaceutically acceptable salts, hydrates, solvates, or polymorphs thereof, and one or more of: (a) at least one agent known to reduce cytokine activity; (b) at least one agent known to reduce abnormal clotting activity; (c) instructions for treating a symptom associated with SARS-CoV-2 infection; or (d) instructions for administering the compound in connection with SARS-CoV-2 infection.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

In one aspect, disclosed herein are kits for treating viral diseases. In a further aspect, the kits can include: (a) a therapeutically effective amount of a tetracycline, (b) a therapeutically effective amount of resveratrol, (c) a therapeutically effective amount of ivermectin, and (d) instructions for treating the viral disease.

In one aspect, in the kits, the tetracycline and resveratrol can be packaged in two separate dosage forms, or can be packaged in the same dosage form. In another aspect, the ivermectin and the resveratrol can be packaged in two separate dosage forms, or can be packaged in the same dosage form.

In one aspect, the kit can include a first dosage form and a second dosage form. Further in this aspect, the first dosage form can include the tetracycline, the resveratrol, and the ivermectin, and the second dosage form can include the tetracycline and the resveratrol. Still further in this aspect, the first dosage form can be administered on from 1 to 4 days per week and the second dosage form can be administered on from 3 to 6 days per week.

In one aspect, when using the kit, the tetracycline can be administered before the resveratrol, after the resveratrol, or simultaneously with the resveratrol. In one aspect, the tetracycline can be doxycycline, CMT-3, CMT-8, or any combination thereof. In another aspect, the resveratrol can be cis-resveratrol, trans-resveratrol, or any combination thereof.

In any of these aspects, the kit can further include an additional therapeutic agent. In one aspect, the additional therapeutic agent can be a painkiller, a cough suppressant, a steroid, an antibody, convalescent plasma, or any combination thereof.

### Methods for Using the Kits and Compositions

The kits and compositions described herein are useful in treating or preventing a viral infection. In one aspect, provided herein is a method for treating or preventing a viral infection, the method including administering the disclosed pharmaceutical compositions or kit to a subject in need thereof. In another aspect, the viral infection can be associated with an influenza virus or a coronavirus. In one aspect, the coronavirus can be SARS CoV-2.

Hospitalizations of COVID-19 patients have led to construction of temporary and field hospitals, reduction in capacity for non-COVID-19 patients in many hospitals, and limited staffing capacities to care for the COVID-19 patients. In a further aspect, effective outpatient treatments can decrease the demand for hospitalization as well as the risk for further transmission of SARS-CoV-2 due to overcrowding and lack of adequate personal protective equipment (PPE). In a further aspect, the disclosed treatments, compounds, and compositions can effectively be used in an outpatient setting. In a still further aspect, the disclosed treatments, compounds, and compositions can be useful in halting the progression of COVID-19 in ambulatory patients prior to the point where hospitalization becomes necessary.

In one aspect, a kit as disclosed herein can be prescribed to patients diagnosed with and/or showing symptoms of COVID-19 along with instructions for use. In a further aspect, supportive therapy can also be prescribed or recommended as needed, including, but not limited to, monitoring of blood oxygen levels and/or supplemental oxygen, anti-platelet and/or anticoagulant therapy, treatment with steroids such as prednisone, inhaled budesonide, prednisolone, or dexamethasone, or the like. In some aspects, additional treatments or altered dosages of kit components can be recommended to patients who are smokers or former smokers.

In one aspect, provided herein is a method for reducing at least one symptom of a viral infection, the method including administering the disclosed pharmaceutical compositions or kit to a subject in need thereof. In another aspect, the viral infection can be associated with an influenza virus or a coronavirus. In one aspect, the coronavirus can be SARS CoV-2.

In another aspect, the at least one symptom can be abnormal clotting activity, low blood oxygen saturation, fever, cough, loss of sense of taste, loss of sense of smell, muscle aches, fatigue, weight loss, increased production of pro-inflammatory cytokines, infiltration of immune cells into lung tissue, an increase in matrix metalloproteinase expression, or any combination thereof.

In one aspect, treatment of bacterial pneumonia is usually difficult, especially when antibiotic-resistant bacteria are involved. In an additional aspect, as current treatments are limited for classical influenza virus or still not readily available for SARS-CoV-2, treatments with already approved medications are being repurposed when a potential effect is observed in preventing viral replication, or if a potential benefit is likely by the use of alternative therapies to modulate the immune system, thus minimizing the disease impact on the individual. In one aspect, and without wishing to be bound by theory, that inhibition of viral replication as well as inhibition of the cytokine storm/ARDS will eventually also be shown to have beneficial effects on other viral (e.g. influenza viruses among others) and bacterial forms of respiratory diseases (e.g. Pseudomonas aeruginosa among others). In a further aspect, it is hypothesized that the use of this approach for management of patients with respiratory diseases extends far beyond COVID-19 and might be applied to many more forms of pneumonias, thereby reducing morbidity and mortality associated with these other diseases.

### ASPECTS

Aspect 1. A pharmaceutical composition comprising therapeutically effective amounts of:
(a) a tetracycline; and
(b) resveratrol or a derivative or variant thereof.

Aspect 2. The pharmaceutical composition of Aspect 1, further comprising:
(c) ivermectin.

Aspect 3. The pharmaceutical composition of Aspect 1 or 2, wherein the tetracycline comprises doxycycline.

Aspect 4. The pharmaceutical composition of any one of Aspects 1-3, wherein the tetracycline comprises a chemically-modified tetracycline.

Aspect 5. The pharmaceutical composition of Aspect 4, wherein the chemically-modified tetracycline comprises 6-demethyl-6-deoxy-4-dedimethylamino-tetracycline (CMT-3), 6a-deoxy-5-hydroxy-4-dedimethylamino-tetracycline (CMT-8), or a combination thereof.

Aspect 6. The pharmaceutical composition of any one of Aspects 1-5, wherein the tetracycline is present in an amount of from about 10 mg to about 300 mg per unit dose.

Aspect 7. The pharmaceutical composition of Aspect 6, wherein the tetracycline is present in an amount of 100 mg per unit dose.

Aspect 8. The pharmaceutical composition of any one of Aspects 1-7, wherein the resveratrol comprises cis-resveratrol, trans-resveratrol, or a combination thereof.

Aspect 9. The pharmaceutical composition of any one of Aspects 1-8, wherein the resveratrol is present in an amount of from about 5 mg to about 3 g per unit dose.

Aspect 10. The pharmaceutical composition of Aspect 9, wherein the resveratrol is present in an amount of about 250 mg per unit dose.

Aspect 11. The pharmaceutical composition of any one of Aspects 2-10, wherein the ivermectin is present in an amount of from about 5 mg to about 1 g.

Aspect 12. The pharmaceutical composition of Aspect 11, wherein the ivermectin is present in an amount of about 18 mg per unit dose.

Aspect 13. The pharmaceutical composition of any one of Aspects 1-12, further comprising at least one excipient.

Aspect 14. The pharmaceutical composition of Aspect 13, wherein the excipient comprises calcium carbonate, sodium carbonate, lactose, calcium phosphate, sodium phosphate, corn starch, alginic acid, gelatin, acacia, magnesium stearate, stearic acid, talc, or any combination thereof.

Aspect 15. The pharmaceutical composition of any one of Aspects 1-14, wherein the pharmaceutical composition comprises an oral dosage form.

Aspect 16. The pharmaceutical composition of Aspect 15, wherein the oral dosage form comprises one or more tablets, capsules, or a combination thereof.

Aspect 17. The pharmaceutical composition of Aspect 16, wherein the one or more tablets, capsules, or combination thereof comprise a coating.

Aspect 18. The pharmaceutical composition of Aspect 17, wherein the coating comprises a copolymer of acrylic acid, methacrylic acid, or an ester thereof; polyvinyl acetate; a fat, an oil, a wax, a fatty alcohol; hydroxypropyl methyl cellulose phthalate; cellulose acetate phthalate, starch acetate phthalate, carboxy methyl cellulose; methyl cellulose phthalate, methyl cellulose succinate; zein; ethyl cellulose; shellac, gluten; ethylcarboxyethyl cellulose; ethacrylate-maleic acid anhydride copolymer; maleic acid anhydride-vinyl methyl ether copolymer; styrol-maleic acid copolymer; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine; glycerol; a phthalic acid ester, ethylphthalyl glycolate, butylphthalylethyl glycolate; propylene glycol, a polyethylene glycol, an adipates, sorbitan monooleate, or any combination thereof.

Aspect 19. The pharmaceutical composition of any one of Aspects 1-18, wherein the pharmaceutical composition is administered from one to three times daily.

Aspect 20. The pharmaceutical composition of any one of Aspects 1-19, wherein the pharmaceutical composition is administered for a period of from 7 days to 60 days.

Aspect 21. The pharmaceutical composition of any one of Aspects 1-20, wherein the tetracycline and the resveratrol are coformulated.

Aspect 22. The pharmaceutical composition of any one of Aspects 1-21, wherein the tetracycline and the resveratrol are separately formulated

Aspect 23. The pharmaceutical composition of Aspect 22, wherein the tetracycline and the resveratrol are co-administered.

Aspect 24. The pharmaceutical composition of any one of Aspects 2-23, wherein the ivermectin and the resveratrol are coformulated.

Aspect 25. The pharmaceutical composition of any one of Aspects 2-23, wherein the ivermectin and the resveratrol are separately formulated.

Aspect 26. The pharmaceutical composition of Aspect 25, wherein the ivermectin and the resveratrol are co-administered.

Aspect 27. A kit for treating a viral or bacterial disease, the kit comprising:
(a) a therapeutically effective amount of a tetracycline;
(b) a therapeutically effective amount of resveratrol;
(c) a therapeutically effective amount of ivermectin; and
(d) instructions for treating the viral or bacterial disease.

Aspect 28. The kit of Aspect 27, further comprising a therapeutically effective amount of acetylsalicylic acid.

Aspect 29. The kit of Aspect 28, wherein the therapeutically effective amount of acetylsalicylic acid is about 325 mg per unit dose.

Aspect 30. The kit of any one of Aspects 27-29, wherein the therapeutically effective amount of the tetracycline is from about 10 mg to about 300 mg per unit dose.

Aspect 31. The kit of Aspect 30, wherein the therapeutically effective amount of the tetracycline is about 100 mg per unit dose.

Aspect 32. The kit of any one of Aspects 27-31, wherein the therapeutically effective amount of resveratrol is from about 5 mg to about 3 g per unit dose.

Aspect 33. The kit of Aspect 32, wherein the therapeutically effective amount of resveratrol is about 250 mg per unit dose.

Aspect 34. The kit of any one of Aspects 27-33, wherein the therapeutically effective amount of ivermectin is from about 5 mg to about 1 g per unit dose.

Aspect 35. The kit of Aspect 34, wherein the therapeutically effective amount of ivermectin is from about 18 mg to about 36 per unit dose.

Aspect 36. The kit of any one of Aspects 27-35, wherein the tetracycline and the resveratrol are packaged in two separate dosage forms.

Aspect 37. The kit of any one of Aspects 27-35, wherein the tetracycline and the resveratrol are packaged in the same dosage form.

Aspect 38. The kit of any one of Aspects 27-37, wherein the ivermectin and the resveratrol are packaged in two separate dosage forms.

Aspect 39. The kit of any one of Aspects 27-37, wherein the ivermectin and the resveratrol are packaged in the same dosage form.

Aspect 40. The kit of any one of Aspects 27-39, wherein the kit comprises a first dosage form and a second dosage form, wherein the first dosage form comprises the tetracycline, the resveratrol, and ivermectin, and wherein the second dosage form comprises the tetracycline and the resveratrol.

Aspect 41. The kit of Aspect 40, wherein the first dosage form is administered on from 1 to 4 days per week and the second dosage form is administered on from 3 to 6 days per week.

Aspect 42. The kit of Aspect 38, wherein the tetracycline is administered simultaneously with the resveratrol.

Aspect 43. The kit of Aspect 38, wherein the tetracycline is administered before or after the resveratrol.

Aspect 44. The kit of Aspect 38, wherein the tetracycline is administered simultaneously with the resveratrol.

Aspect 45. The kit of any one of Aspects 27-44, wherein the tetracycline comprises doxycycline, CMT-3, CMT-8, or any combination thereof.

Aspect 46. The kit of any one of Aspects 27-45, wherein the resveratrol comprises cis-resveratrol, trans-resveratrol, or any combination thereof.

Aspect 47. The kit of any one of Aspects 27-46, further comprising an additional therapeutic agent.

Aspect 48. The kit of Aspect 47, wherein the additional therapeutic agent comprises a painkiller, a cough suppressant, a steroid, an antibody, convalescent plasma, or any combination thereof.

Aspect 49. A method for reducing at least one symptom of a viral infection or bacterial infection, the method comprising administering the pharmaceutical composition of any of Aspects 1-26 or the kit of any of Aspects 27-48 to a subject in need thereof.

Aspect 50. A method for preventing a viral infection, the method comprising administering the pharmaceutical composition of any of Aspects 1-26 or the kit of any of Aspects 27-48 to a subject in need thereof.

Aspect 51. The method of Aspect 49 or 50, wherein the viral infection is an influenza virus infection, a coronavirus infection, or a respiratory syncytial virus infection.

Aspect 52. The method of Aspect 51, wherein the coronavirus comprises SARS-CoV-2.

Aspect 53. The method of Aspect 49, wherein the bacterial infection is a bacterial pneumonia infection.

Aspect 54. The method of any one of Aspects 49, 50, or 52, wherein the at least one symptom comprises abnormal clotting activity, low blood oxygen saturation, fever, cough, loss of sense of taste, loss of sense of smell, muscle aches, fatigue, weight loss, increased production of pro-inflammatory cytokines, infiltration of immune cells into lung tissue, an increase in matrix metalloproteinase expression, or any combination thereof.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Dosage Packs and Dosage Regimens

Exemplary dosage regimens are provided in the tables below.

An exemplary dose pack for prophylactic care and prevention of COVID-19 “long haul” syndrome is provided in Table 1 below.

TABLE 1
Exemplary Prophylactic and “Long Haul” Prevention Dosage Regimen

| Week | Day 1 | Day 2 | Day 3 | Day 4 | Day 5 | Day 6 | Day 7 |
|---|---|---|---|---|---|---|---|
| 1 | Ivermectin^{a} | CM Doxycycline | CM Doxycycline | CM Doxycycline | CM Doxycycline | CM Doxycycline | CM Doxycycline |
| | CM Doxycycline^{b} | Resveratrol | Resveratrol | Resveratrol | Resveratrol | Resveratrol | Resveratrol |
| | Resveratrol^{c} | | | | | | |
| 2 | Ivermectin | CM Doxycycline | CM Doxycycline | CM Doxycycline | CM Doxycycline | CM Doxycycline | CM Doxycycline |
| | CM Doxycycline | Resveratrol | Resveratrol | Resveratrol | Resveratrol | Resveratrol | Resveratrol |
| | Resveratrol | | | | | | |
| 3 | Ivermectin | CM Doxycycline | CM Doxycycline | CM Doxycycline | CM Doxycycline | CM Doxycycline | CM Doxycycline |
| | CM Doxycycline | Resveratrol | Resveratrol | Resveratrol | Resveratrol | Resveratrol | Resveratrol |
| | Resveratrol | | | | | | |
| 4 | Ivermectin | CM Doxycycline | CM Doxycycline | CM Doxycycline | CM Doxycycline | CM Doxycycline | CM Doxycycline |
| | CM Doxycycline | Resveratrol | Resveratrol | Resveratrol | Resveratrol | Resveratrol | Resveratrol |
| | Resveratrol | | | | | | |

^{a}Ivermectin Dosage: 18 mg/week or 36 mg/week
^{b}CM-Doxycycline Dosage: 100 mg/day
^{c}Resveratrol Dosage: 250 mg/day

The exemplary dosage regimen in Table 1 can be provided in a dosage pack containing combination pills and/or individual pills to complete the dosage indicated.

A second exemplary dose pack for treatment of COVID-19 is provided in Table 2 below:

TABLE 2
Exemplary Two Week Combination Dosage Regimen

| Week | Day 1 | Day 2 | Day 3 | Day 4 | Day 5 | Day 6 | Day 7 |
|---|---|---|---|---|---|---|---|
| 1, AM | Ivermectin^{a} | CM Doxycycline | Ivermectin | CM Doxycycline | Ivermectin | CM Doxycycline | Ivermectin |
| | CM Doxycycline^{b} | Resveratrol | CM Doxycycline | Resveratrol | CM Doxycycline | Resveratrol | CM Doxycycline |
| | Resveratrol^{c} | | Resveratrol | | Resveratrol | | Resveratrol |
| 1, PM | CM Doxycycline | CM Doxycycline | CM Doxycycline | CM Doxycycline | CM Doxycycline | CM Doxycycline | CM Doxycycline |
| | Resveratrol | Resveratrol | Resveratrol | Resveratrol | Resveratrol | Resveratrol | Resveratrol |
| 2, AM | Ivermectin | CM Doxycycline | Ivermectin | CM Doxycycline | Ivermectin | CM Doxycycline | Ivermectin |
| | CM Doxycycline | Resveratrol | CM Doxycycline | Resveratrol | CM Doxycycline | Resveratrol | CM Doxycycline |
| | Resveratrol | | Resveratrol | | Resveratrol | | Resveratrol |
| 2, PM | CM Doxycycline | CM Doxycycline | CM Doxycycline | CM Doxycycline | CM Doxycycline | CM Doxycycline | CM Doxycycline |
| | Resveratrol | Resveratrol | Resveratrol | Resveratrol | Resveratrol | Resveratrol | Resveratrol |

^{a}Ivermectin Dosage: 9 mg/pill (18 mg/day, where present)
^{b}CM-Doxycycline Dosage: 50 mg/pill (100 mg/day)
^{c}Resveratrol Dosage: 125 mg/pill (250 mg/day)

In one example, the dosage regimen in Table 1 (i.e., 4 week treatment) can be particularly useful for preventing infection in individuals at high risk for infection, such as, for example, health care workers, other persons with jobs requiring high public exposure in areas of high community transmission, or individuals who are immunocompromised.

In another example, the dosage regimen in Table 2 (i.e., 2 week treatment) can be particularly useful for individuals who present with disease symptoms in addition to positive PCR test results or positive antigen test results for COVID-19. When these subjects recover, if “long haul” symptoms are present, the dosage regimen in Table 2 can be followed by the dosage regimen in Table 1.

### Example 2: Effects of Selected Compounds on PMN-Driven Inflammatory Responses

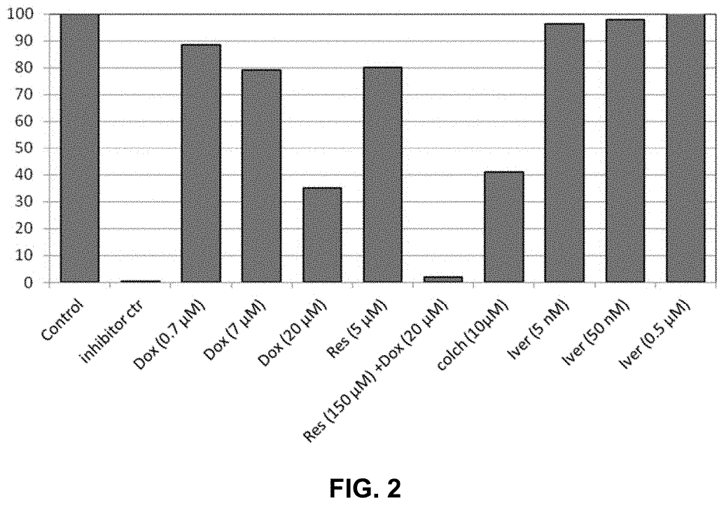

Pilot investigations were carried out focusing on the ability of doxycycline, resveratrol, and ivermectin to inhibit MMP activity. As anticipated, doxycycline along expressed clear inhibitory effects of MMP activity as seen in FIG. 2. It also appeared that resveratrol alone inhibited MMP activity but to a much more modest extent compared to doxycycline. However, when both doxycycline and resveratrol were combined, a completely unanticipated and synergistic effect occurred where there was virtually complete inhibition of MMP activity in the assay system used.

Inhibition of MMP9 was determined using the MMP9 inhibitor screening assay kit (Fluorometric) from Abcam (Cat#ab139449) according to the manufacturer's instructions. Unquenching of a fluorogenic peptide by MMP9 was monitored for 10 minutes in the presence or absence of inhibitory compounds, using a fluorescent microplate reader (Ex/Em=328/420). Reaction velocity (V) in RFU/minute was used to determine the % of remaining enzymatic activity, relative to untreated control, according to the following equation: (V_{inhibitor}/V_{control})×100 (FIG. 2).

Ivermectin has not been shown to interfere with these MMP-inhibiting effects.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Alexander P E, et al. Early Multidrug Outpatient Treatment of SARS-CoV-2 Infection (COVID-19) and Reduced Mortality Among Nursing Home Residents. medRxiv. 2021: 2021.01.28.21250706.
2. Alqahtani J S, et al. Prevalence, Severity and Mortality associated with COPD and Smoking in patients with COVID-19: A Rapid Systematic Review and Meta-Analysis. PLoS One. 2020; 15 (5): e0233147.
3. Andrade E F, et al. Can Resveratrol Treatment Control the Progression of Induced Periodontal Disease? A Systematic Review and Meta-Analysis of Preclinical Studies. Nutrients. 2019; 11 (5).
4. Bao H, et al. Angiotensin-Converting Enzyme 2 Inhibits Apoptosis of Pulmonary Endothelial Cells During Acute Lung Injury Through Suppressing MiR-4262. Cell Physiol Biochem. 2015; 37 (2): 759-67.
5. Barnes B J, et al. Targeting potential drivers of COVID-19: Neutrophil extracellular traps. J Exp Med. 2020; 217 (6).
6. Berntsson E, et al. Etiology of community-acquired pneumonia in out-patients. Eur J Clin Microbiol. 1986; 5 (4): 446-7.
7. Bhaskar S, et al. Cytokine Storm in COVID-19-Immunopathological Mechanisms, Clinical Considerations, and Therapeutic Approaches: The REPROGRAM Consortium Position Paper. Front Immunol. 2020; 11: 1648.
8. Caly L, et al. The FDA-approved drug ivermectin inhibits the replication of SARS-CoV-2 in vitro. Antiviral Res. 2020; 178: 104787.
9. Campagna M, et al. Antiviral activity of resveratrol. Biochem Soc Trans. 2010; 38 (Pt 1): 50-3.
10. Casper R F, et al. Resveratrol has antagonist activity on the aryl hydrocarbon receptor: implications for prevention of dioxin toxicity. Mol Pharmacol. 1999; 56 (4): 784-90.
11. Cazalis J, et al. Tetracyclines and chemically modified tetracycline-3 (CMT-3) modulate cytokine secretion by lipopolysaccharide-stimulated whole blood. Inflammation. 2009; 32 (2): 130-7.
12. Choi B, et al. Dipeptidyl Peptidase-4 Induces Aortic Valve Calcification by Inhibiting Insulin-Like Growth Factor-1 Signaling in Valvular Interstitial Cells. Circulation. 2017; 135 (20): 1935-50.
13. Choi H J, et al. Antiviral activity of raoulic acid from Raoulia australis against Picornaviruses. Phytomedicine. 2009; 16 (1): 35-9.
14. Cianfrocca M, et al. Matrix metalloproteinase inhibitor COL-3 in the treatment of AIDS-related Kaposi's sarcoma: a phase I AIDS malignancy consortium study. J Clin Oncol. 2002; 20 (1): 153-9.
15. Cirano F R, et al. Resveratrol and insulin association reduced alveolar bone loss and produced an antioxidant effect in diabetics rats. J Periodontol. 2020.
16. Conforti C, et al. Doxycycline, a widely used antibiotic in dermatology with a possible anti-inflammatory action against IL-6 in COVID-19 outbreak. Dermatol Ther. 2020: e13437.
17. Correa M G, et al. Systemic treatment with resveratrol and/or curcumin reduces the progression of experimental periodontitis in rats. J Periodontal Res. 2017; 52 (2): 201-9.
18. Curylofo-Zotti F A, et al. Differential effects of natural Curcumin and chemically modified curcumin on inflammation and bone resorption in model of experimental periodontitis. Arch Oral Biol. 2018; 91: 42-50.
19. de Almeida Brandão D, et al. Dose-response assessment of chemically modified curcumin in experimental periodontitis. J Periodontol. 2019; 90 (5): 535-45.
20. DeDiego M L, et al. Inhibition of NF-κB-mediated inflammation in severe acute respiratory syndrome coronavirus-infected mice increases survival. J Virol. 2014; 88 (2): 913-24.
21. Deng J, et al. Chemically-Modified Curcumin 2.24: A Novel Systemic Therapy for Natural Periodontitis in Dogs. J Exp Pharmacol. 2020; 12: 47-60.
22. Dezube B J, et al. Randomized phase II trial of matrix metalloproteinase inhibitor COL-3 in AIDS-related Kaposi's sarcoma: an AIDS Malignancy Consortium Study. J Clin Oncol. 2006; 24 (9): 1389-94.
23. Domingo P, et al. The four horsemen of a viral Apocalypse: The pathogenesis of SARS-CoV-2 infection (COVI D-19). EBioMedicine. 2020; 58: 102887.
24. Emingil G, et al. The effect of adjunctive subantimicrobial dose doxycycline therapy on GCF EMMPRIN levels in chronic periodontitis. J Periodontol. 2008; 79 (3): 469-76.
25. Eybl V, et al. Comparative study of natural antioxidants—curcumin, resveratrol and melatonin—in cadmium-induced oxidative damage in mice. Toxicology. 2006; 225 (2-3): 150-6.
26. Fine N, et al. Periodontal Inflammation Primes the Systemic Innate Immune Response. J Dent Res. 2020: 22034520963710.
27. Fine N, et al. The Neutrophil: Constant Defender and First Responder. Front Immunol. 2020; 11: 571085.
28. Golub L M, et al. A chemically modified nonantimicrobial tetracycline (CMT-8) inhibits gingival matrix metalloproteinases, periodontal breakdown, and extra-oral bone loss in ovariectomized rats. Ann N Y Acad Sci. 1999; 878: 290-310.
29. Golub L M, et al. A non-antibacterial chemically-modified tetracycline inhibits mammalian collagenase activity. J Dent Res. 1987; 66 (8): 1310-4.
30. Golub L M, et al. Host modulation with tetracyclines and their chemically modified analogues. Curr Opin Dent. 1992; 2:80-90.
31. Golub L M, et al. Modulation of the host response in the treatment of periodontitis. Dent Today. 1998; 17 (10): 102-6, 8-9.
32. Golub L M, et al. Periodontal therapeutics: Current host-modulation agents and future directions. Periodontol 2000. 2020; 82 (1): 186-204.
33. Golub L M, et al. Tetracyclines inhibit connective tissue breakdown: new therapeutic implications for an old family of drugs. Crit Rev Oral Biol Med. 1991; 2 (3): 297-321.
34. Golub L M, et al. Treating periodontal diseases by blocking tissue-destructive enzymes. J Am Dent Assoc. 1994; 125 (2): 163-9; discussion 9-71.
35. Gouda A S, et al. Snake venom-derived bradykinin-potentiating peptides: A promising therapy for COVID-19? Drug Dev Res. 2020.
36. Greenwald R, et al. Biologic properties of non-antibiotic, chemically modified tetracyclines (CMTs): a structured, annotated bibliography. Curr Med Chem. 2001; 8 (3): 237-42.
37. Griffin M O, et al. Tetracyclines: a pleitropic family of compounds with promising therapeutic properties. Review of the literature. Am J Physiol Cell Physiol. 2010; 299 (3): C539-48.
38. Gu Y, et al. Non-antibacterial tetracycline formulations: clinical applications in dentistry and medicine. J Oral Microbiol. 2012; 4.
39. Haga S, et al. Modulation of TNF-alpha-converting enzyme by the spike protein of SARS-CoV and ACE2 induces TNF-alpha production and facilitates viral entry. Proc Natl Acad Sci U S A. 2008; 105 (22): 7809-14.
40. Henehan M, et al. Doxycycline as an anti-inflammatory agent: updates in dermatology. J Eur Acad Dermatol Venereol. 2017; 31 (11): 1800-8.
41. Holmes S G, et al. Chemically modified tetracyclines act through multiple mechanisms directly on osteoclast precursors. Bone. 2004; 35 (2): 471-8.
42. Holmes-McNary M, et al. Chemopreventive properties of trans-resveratrol are associated with inhibition of activation of the IkappaB kinase. Cancer Res. 2000; 60 (13): 3477-83.
43. Hoyt J C, et al. Doxycycline modulates nitric oxide production in murine lung epithelial cells. J Immunol. 2006; 176 (1): 567-72.
44. Hu B, et al. The cytokine storm and COVI D-19. J Med Virol. 2020.
45. Huang C, et al. Clinical features of patients infected with 2019 novel coronavirus in Wuhan, China. Lancet. 2020; 395 (10223): 497-506.
46. Huang F C, et al. Anti-inflammatory effect of resveratrol in human coronary arterial endothelial cells via induction of autophagy: implication for the treatment of Kawasaki disease. BMC Pharmacol Toxicol. 2017; 18 (1): 3.
47. Hussman J P. Cellular and Molecular Pathways of COVID-19 and Potential Points of Therapeutic Intervention. Front Pharmacol. 2020; 11: 1169.
48. Ikeda E, et al. Resveratrol derivative-rich melinjo seed extract induces healing in a murine model of established periodontitis. J Periodontol. 2018; 89 (5) :586-95.
49. Imai Y, et al. Angiotensin-converting enzyme 2 protects from severe acute lung failure. Nature. 2005; 436 (7047): 112-6.
50. Jia H. Pulmonary Angiotensin-Converting Enzyme 2 (ACE2) and Inflammatory Lung Disease. Shock. 2016; 46 (3): 239-48.
51. Kirkwood K, et al. Chemically modified tetracyclines selectively inhibit IL-6 expression in osteoblasts by decreasing mRNA stability. Biochem Pharmacol. 2003; 66 (9): 1809-19.
52. Kong M Y, et al. Matrix Metalloproteinase-9 Mediates RSV Infection in Vitro and in Vivo. Viruses. 2015; 7 (8): 4230-53.
53. Kumar N, et al. NF-kappaB signaling differentially regulates influenza virus RNA synthesis. J Virol. 2008; 82 (20): 9880-9.
54. Leonard S S, et al. Resveratrol scavenges reactive oxygen species and effects radical-induced cellular responses. Biochem Biophys Res Commun. 2003; 309 (4): 1017-26.
55. Limagne E, et al. Resveratrol Interferes with IL113-Induced Pro-Inflammatory Paracrine Interaction between Primary Chondrocytes and Macrophages. Nutrients. 2016; 8 (5).
56. Lin S C, et al. Effective inhibition of MERS-CoV infection by resveratrol. BMC Infect Dis. 2017; 17 (1): 144.
57. Lin Y G, et al. Curcumin inhibits tumor growth and angiogenesis in ovarian carcinoma by targeting the nuclear factor-kappaB pathway. Clin Cancer Res. 2007; 13 (11): 3423-30.
58. Lindeman J H, et al. Clinical trial of doxycycline for matrix metalloproteinase-9 inhibition in patients with an abdominal aneurysm: doxycycline selectively depletes aortic wall neutrophils and cytotoxic T cells. Circulation. 2009; 119 (16): 2209-16.
59. Llavaneras A, et al. A combination of a chemically modified doxycycline and a bisphosphonate synergistically inhibits endotoxin-induced periodontal breakdown in rats. J Periodontol. 2001; 72 (8): 1069-77.
60. Lokeshwar B L. Chemically modified non-antimicrobial tetracyclines are multifunctional drugs against advanced cancers. Pharmacol Res. 2011; 63 (2): 146-50.
61. Lu R, et al. Genomic characterisation and epidemiology of 2019 novel coronavirus: implications for virus origins and receptor binding. Lancet. 2020; 395 (10224): 565-74.
62. Mahmudpour M, et al. COVID-19 cytokine storm: The anger of inflammation. Cytokine. 2020; 133:155151.
63. Malek A E, et al. Doxycycline as a potential partner of COVID-19 therapies. IDCases. 2020; 21: e00864.
64. Mandell L A, et al. Canadian guidelines for the initial management of community-acquired pneumonia: an evidence-based update by the Canadian Infectious Diseases Society and the Canadian Thoracic Society. The Canadian Community-Acquired Pneumonia Working Group. Clin Infect Dis. 2000; 31 (2): 383-421.
65. Marrie T J, et al. Ambulatory patients with community-acquired pneumonia: the frequency of atypical agents and clinical course. Am J Med. 1996; 101 (5): 508-15.
66. McCullers J A. Effect of antiviral treatment on the outcome of secondary bacterial pneumonia after influenza. J Infect Dis. 2004; 190 (3): 519-26.
67. McCullough PA, et al. Multifaceted highly targeted sequential multidrug treatment of early ambulatory high-risk SARS-CoV-2 infection (COVID-19). Rev Cardiovasc Med. 2020; 21 (4): 517-30.
68. McNamara T F, et al; Non-antibacterial tetracycline compositions possessing anti-collagenolytic properties and methods of preparing and using same patent U.S. Pat. No. 4,704,383A. 1987.
69. McNamara T F, et al, editors. The Synthesis and Characterization of a Non-antibacterial Chemically Modified Tetracycline. AADR; 1986: J Dent Res.
70. Menon V P, et al. Antioxidant and anti-inflammatory properties of curcumin. Adv Exp Med Biol. 2007; 595: 105-25.
71. Moran C S, et al. Resveratrol Inhibits Growth of Experimental Abdominal Aortic Aneurysm Associated With Upregulation of Angiotensin-Converting Enzyme 2. Arterioscler Thromb Vasc Biol. 2017; 37 (11): 2195-203.
72. Muir R, et al. Observations ON INFLUENZA AND ITS COMPLICATIONS. Br Med J. 1919; 1 (3027): 3-5.
73. Nallusamy S, et al. Shortlisting Phytochemicals Exhibiting Inhibitory Activity against Major Proteins of SARS-CoV-2 through Virtual Screening 2020.
74. Oliveira Andrade J M, et al. Cross talk between angiotensin-(1-7)/Mas axis and sirtuins in adipose tissue and metabolism of high-fat feed mice. Peptides. 2014; 55:158-65.
75. Phillips J M, et al. Neurovirulent Murine Coronavirus JHM.SD Uses Cellular Zinc Metalloproteases for Virus Entry and Cell-Cell Fusion. J Virol. 2017; 91 (8).
76. Potey P M, et al. Neutrophils in the initiation and resolution of acute pulmonary inflammation: understanding biological function and therapeutic potential. J Pathol. 2019; 247 (5): 672-85.
77. Procter B C, et al. Clinical outcomes after early ambulatory multidrug therapy for high-risk SARS-CoV-2 (COVID-19) infection. Rev Cardiovasc Med. 2020; 21 (4): 611-4.
78. Quah P, et al, Mortality rates of patients with COVID-19 in the intensive care unit: a systematic review of the emerging literature. Crit Care. 2020; 24 (1): 285.
79. Ragab D, et al. The COVID-19 Cytokine Storm; What We Know So Far. Front Immunol. 2020; 11: 1446.
80. Ramdani L H, et al. Potential therapeutic effects of Resveratrol against SARS-CoV-2. Acta Virol. 2020.
81. Reyes L, et al. A type I IFN, prothrombotic hyperinflammatory neutrophil signature is distinct for COVI D-19 ARDS. medRxiv. 2021: 2020.09.15.20195305.
82. Ribeiro F V, et al. Resveratrol Inhibits Periodontitis-Related Bone Loss in Rats Subjected to Cigarette Smoke Inhalation. J Periodontol. 2017; 88 (8): 788-98.
83. Richards C, et al. Antimicrobial and non-antimicrobial tetracyclines in human cancer trials. Pharmacol Res. 2011; 63 (2): 151-6.
84. Roche J A, et al. A hypothesized role for dysregulated bradykinin signaling in COVID-19 respiratory complications. FASEB J. 2020; 34 (6): 7265-9.
85. Roy S K, et al. Chemically modified tetracycline 3 prevents acute respiratory distress syndrome in a porcine model of sepsis+ischemia/reperfusion-induced lung injury. Shock. 2012; 37 (4) : 424-32.
86. Roy S K, et al. Jack of all trades: pleiotropy and the application of chemically modified tetracycline-3 in sepsis and the acute respiratory distress syndrome (ARDS). Pharmacol Res. 2011; 64 (6): 580-9.
87. Sadowsky D, et al. Impact of chemically-modified tetracycline 3 on intertwined physiological, biochemical, and inflammatory networks in porcine sepsis/ARDS. Int J Burns Trauma. 2015; 5 (1): 22-35.
88. Sandler C, et al. Chemically modified tetracyclines induce apoptosis in cultured mast cells. Int Immunopharmacol. 2005; 5 (11): 1611-21.
89. Santa-Cecilia F V, et al. Doxycycline Suppresses Microglial Activation by Inhibiting the p38 MAPK and NF-kB Signaling Pathways. Neurotox Res. 2016; 29 (4): 447-59.
90. Sargiacomo C, et al. COVID-19 and chronological aging: senolytics and other anti-aging drugs for the treatment or prevention of corona virus infection? Aging (Albany N.Y.). 2020; 12 (8): 6511-7.
91. Sodeman W A, et al. Instructions for geriatric patients. Philadelphia, Pa.: Elsevier Saunders; 2005.
92. Sodhi C P, et al. Attenuation of pulmonary ACE2 activity impairs inactivation of des-Arg. Am J Physiol Lung Cell Mol Physiol. 2018; 314 (1): L17-L31.
93. Soy M, et al. Cytokine storm in COVID-19: pathogenesis and overview of anti-inflammatory agents used in treatment. Clin Rheumatol. 2020; 39 (7): 2085-94.
94. Steinberg J, et al. Chemically modified tetracycline prevents the development of septic shock and acute respiratory distress syndrome in a clinically applicable porcine model. Shock. 2005; 24 (4): 348-56.
95. Sun X, et al. Cytokine storm intervention in the early stages of COVID-19 pneumonia. Cytokine Growth Factor Rev. 2020; 53: 38-42.
96. Švajger U, et al. Anti-inflammatory effects of resveratrol and its potential use in therapy of immune-mediated diseases. Int Rev Immunol. 2012; 31 (3): 202-22.
97. to Velthuis A J, et al. Zn(2+) inhibits coronavirus and arterivirus RNA polymerase activity in vitro and zinc ionophores block the replication of these viruses in cell culture. PLoS Pathog. 2010; 6 (11): e1001176.
98. Teijaro J R, et al. Mapping the innate signaling cascade essential for cytokine storm during influenza virus infection. Proc Natl Acad Sci U S A. 2014; 111 (10): 3799-804.
99. Tiao M M, et al. Resveratrol ameliorates maternal and post-weaning high-fat diet-induced nonalcoholic fatty liver disease via renin-angiotensin system. Lipids Health Dis. 2018; 17 (1): 178.
100. Trachtman H, et al. Chemically modified tetracyclines inhibit inducible nitric oxide synthase expression and nitric oxide production in cultured rat mesangial cells. Biochem Biophys Res Commun. 1996; 229 (1): 243-8.
101. Truong V L, et al. Role of resveratrol in regulation of cellular defense systems against oxidative stress. Biofactors. 2018; 44 (1): 36-49.
102. Tyrrell D A. The pulmonary complications of influenza as seen in Sheffield in 1949. Q J Med. 1952; 21 (83): 291-306.
103. US Centers for Disease Control and Prevention, Vaccine and Other Medical Countermeasures <https://www.cdc.gov/flu/pandemic-resources/planning-preparedness/vaccine-medical-countermeasures.html> Accessed Dec. 11, 2020.
104. van de Veerdonk F L, et al. Kallikrein-kinin blockade in patients with COVID-19 to prevent acute respiratory distress syndrome. Elife. 2020; 9.
105. Vankadari N, et al. Emerging WuHan (COVID-19) coronavirus: glycan shield and structure prediction of spike glycoprotein and its interaction with human CD26. Emerg Microbes Infect. 2020; 9 (1): 601-4.
106. Verdoni L, et al. An outbreak of severe Kawasaki-like disease at the Italian epicentre of the SARS-CoV-2 epidemic: an observational cohort study. Lancet. 2020; 395 (10239): 1771-8.
107. Voronov I, et al. Inhibition of osteoclast differentiation by polycyclic aryl hydrocarbons is dependent on cell density and RANKL concentration. Biochem Pharmacol. 2005; 70 (2): 300-7.
108. Wagstaff K M, et al. Ivermectin is a specific inhibitor of importin α/β-mediated nuclear import able to inhibit replication of HIV-1 and dengue virus. Biochem J. 2012; 443 (3): 851-6.
109. Wan S, et al. Relationships among lymphocyte subsets, cytokines, and the pulmonary inflammation index in coronavirus (COVID-19) infected patients. Br J Haematol. 2020; 189 (3): 428-37.
110. Wang D, et al. Clinical Characteristics of 138 Hospitalized Patients With 2019 Novel Coronavirus-Infected Pneumonia in Wuhan, China. JAMA. 2020.
111. Wang Q W, et al. Anti-influenza A virus activity of rhein through regulating oxidative stress, TLR4, Akt, MAPK, and NF-κB signal pathways. PLoS One. 2018; 13 (1): e0191793.
112. Wong Y, et al. Synthesis of 4′-ester analogs of resveratrol and their evaluation in malignant melanoma and pancreatic cell lines. Bioorg Med Chem Lett. 2010; 20 (3): 1198-201.
113. Wu C, et al. Analysis of therapeutic targets for SARS-CoV-2 and discovery of potential drugs by computational methods. Acta Pharm Sin B. 2020; 10 (5): 766-88.
114. Yang C M, et al. Resveratrol inhibits BK-induced COX-2 transcription by suppressing acetylation of AP-1 and NF-KB in human rheumatoid arthritis synovial fibroblasts. Biochem Pharmacol. 2017; 132: 77-91.
115. Yang H, et al. Interleukin-1 promotes coagulation, which is necessary for protective immunity in the lung against Streptococcus pneumoniae infection. J Infect Dis. 2013; 207 (1): 50-60.
116. Yang S N Y, et al. The broad spectrum antiviral ivermectin targets the host nuclear transport importin α/β1 heterodimer. Antiviral Res. 2020; 177:104760.
117. Yates P A, et al. Doxycycline treatment of high-risk COVID-19-positive patients with comorbid pulmonary disease. Ther Adv Respir Dis. 2020; 14: 1753466620951053.
118. Ye Q, et al. The pathogenesis and treatment of the ‘Cytokine Storm’ in COVID-19. J Infect. 2020; 80 (6) : 607-13.
119. Yu Z, et al. HPLC determination of a chemically modified nonantimicrobial tetracycline: biological implications. Biochem Med Metab Biol. 1992; 47 (1): 10-20.
120. Zang N, et al. Resveratrol-mediated gamma interferon reduction prevents airway inflammation and airway hyperresponsiveness in respiratory syncytial virus-infected immunocompromised mice. J Virol. 2011; 85 (24): 13061-8.
121. Zhang J J, et al. Clinical characteristics of 140 patients infected with SARS-CoV-2 in Wuhan, China. Allergy. 2020; 75 (7): 1730-41.
122. Zhang Y, et al. Design, synthesis and biological activity of new polyenolic inhibitors of matrix metalloproteinases: a focus on chemically-modified curcumins. Curr Med Chem. 2012; 19 (25): 4348-58.
123. Zhou F, et al. Clinical course and risk factors for mortality of adult inpatients with COVID-19 in Wuhan, China: a retrospective cohort study. Lancet. 2020; 395 (10229): 1054-62.
124. Zhou M. Participation in international human rights NGOs: The effect of democracy and state capacity. Social Science Research. 2012; 41 (5): 1254-74.
125. Zhou X, et al. A tetracycline analog improves acute respiratory distress syndrome survival in an ovine model. Ann Thorac Surg. 2010; 90 (2): 419-26.

1. A pharmaceutical composition comprising therapeutically an effective amount of:
(a) a tetracycline; and
(b) resveratrol or a derivative or variant thereof.

2. The pharmaceutical composition of claim 1, further comprising:
(c) ivermectin.

3. The pharmaceutical composition of claim 1, wherein the tetracycline comprises doxycycline.

4. The pharmaceutical composition of claim 1, wherein the tetracycline comprises a chemically-modified tetracycline.

5. The pharmaceutical composition of claim 4, wherein the chemically-modified tetracycline comprises 6-demethyl-6-deoxy-4-dedimethylamino-tetracycline (CMT-3), 6a-deoxy-5-hydroxy-4-dedimethylamino-tetracycline (CMT-8), or a combination thereof.

6. The pharmaceutical composition of claim 1, wherein the tetracycline is present in an amount of from about 10 mg to about 300 mg per unit dose.

7. The pharmaceutical composition of claim 1, wherein the resveratrol comprises cis-resveratrol, trans-resveratrol, or a combination thereof.

8. The pharmaceutical composition of claim 2, wherein the ivermectin is present in an amount of from about 5 mg to about 1 g.

9. The pharmaceutical composition of claim 1, further comprising at least one excipient.

10. The pharmaceutical composition of claim 1, wherein the pharmaceutical composition comprises an oral dosage form, wherein the oral dosage form comprises one or more tablets, capsules, or a combination thereof.

11. The pharmaceutical composition of claim 10, wherein the one or more tablets, capsules, or combination thereof comprise a coating.

12. A kit for treating a viral or bacterial disease, the kit comprising:
(a) a therapeutically effective amount of a tetracycline;
(b) a therapeutically effective amount of resveratrol;
(c) a therapeutically effective amount of ivermectin; and
(d) instructions for treating the viral or bacterial disease.

13. The kit of claim 12, further comprising a therapeutically effective amount of acetylsalicylic acid.

14. The kit of claim 12, wherein the therapeutically effective amount of the tetracycline is from about 10 mg to about 300 mg per unit dose.

15. The kit of claim 12, wherein the therapeutically effective amount of resveratrol is from about 5 mg to about 3 g per unit dose.

16. The kit of claim 12, wherein the therapeutically effective amount of ivermectin is from about 5 mg to about 1 g per unit dose.

17. The kit of claim 12, wherein the tetracycline and the resveratrol are packaged in two separate dosage forms.

18. The kit of claim 12, wherein the tetracycline and the resveratrol are packaged in the same dosage form.

19. The kit of claim 12, wherein the ivermectin and the resveratrol are packaged in two separate dosage forms.

20. The kit of claim 12, wherein the ivermectin and the resveratrol are packaged in the same dosage form.

21. The kit of claim 12, wherein the kit comprises a first dosage form and a second dosage form, wherein the first dosage form comprises the tetracycline, the resveratrol, and ivermectin, and wherein the second dosage form comprises the tetracycline and the resveratrol.

22. The kit of claim 12, wherein the tetracycline comprises doxycycline, CMT-3, CMT-8, or any combination thereof.

23. The kit of claim 12, wherein the resveratrol comprises cis-resveratrol, trans-resveratrol, or any combination thereof.

24. The kit of claim 12, further comprising an additional therapeutic agent, wherein the additional therapeutic agent comprises a painkiller, a cough suppressant, a steroid, an antibody, convalescent plasma, or any combination thereof.

25. A method for reducing at least one symptom of a viral infection or bacterial infection, the method comprising administering the pharmaceutical composition of any of claim 1 to a subject in need thereof.

26. The method of claim 25, wherein the viral infection is an influenza virus infection, a coronavirus infection, or a respiratory syncytial virus infection.

27. The method of claim 26, wherein the coronavirus comprises SARS-CoV-2.

28. The method of claim 25, wherein the bacterial infection is a bacterial pneumonia infection.

29. The method of claim 25, wherein the at least one symptom comprises abnormal clotting activity, low blood oxygen saturation, fever, cough, loss of sense of taste, loss of sense of smell, muscle aches, fatigue, weight loss, increased production of pro-inflammatory cytokines, infiltration of immune cells into lung tissue, an increase in matrix metalloproteinase expression, or any combination thereof.

30. A method for reducing at least one symptom of a viral infection or bacterial infection, the method comprising administering the kit of claim 12 to a subject in need thereof.
